(19) United States
(12) Patent Application Publication
(10) Pub. No.: US 2007/0292888 A1
(43) Pub. Date: Dec. 20, 2007

(54) Methods for Measuring Enzyme Activities in Cells, Blood, Tumors and Tissues

(75) Inventor: May Han, Brookline, MA (US)

Correspondence Address: ROPES & GRAY LLP;PATENT DOCKETING 39/361, 1211 AVENUE OF THE AMERICAS, NEW YORK, NY, 10036-8704, US

(73) Assignee: GEN-PATH PHARMACEUTICALS, INC., Cambridge, MA (US)

(21) Appl. No.: 10/594,194

(22) PCT Filed: Mar. 23, 2005
(86) PCT No.: PCT/US05/09759

§ 371 Date: Apr. 27, 2007

Related U.S. Application Data
(60) Provisional application No. 60/556,281, filed on Mar. 24, 2004.

(51) Int. Cl. G01N 33/573; C12Q 1/32; C12Q 1/48; G01N 33/53

(52) U.S. Cl. 435/7.1; 435/15; 435/26; 435/4

(57) ABSTRACT

The invention provides improved methods for measuring enzymatic activities involved in the oxidative and non-oxidative pentose phosphate pathways, and other thiamine pyrophosphate containing enzymes, which are commonly elevated in tumor cells. The assays of the invention are useful for optimizing therapeutic dosing and scheduling of drugs acting on the pentose phosphate pathways by sampling and monitoring enzymatic levels in a treated patient over time. Finally, the assays are useful for identifying and characterizing compounds having beneficial therapeutic effects.

### CROSS-REFERENCE TO RELATED APPLICATION

This application claims priority to U.S. Provisional Application: Ser. No. 60/556,281 filed Mar. 24, 2004, which is incorporated herein by reference in its entirety.

### FIELD OF THE INVENTION

The invention is directed to improved and high through-put methods for measuring the activities of enzymes involved in oxidative and non-oxidative pentose phosphate pathways and other thiamine pyrophosphate containing enzymes which are commonly elevated in tumor cells. The assays of the invention are useful for optimizing therapeutic dosing and scheduling of drugs acting on the pentose phosphate pathways by sampling and monitoring enzymatic levels in a treated patient over time. Moreover, the assays are useful for identifying and characterizing compounds having potentially beneficial therapeutic effects.

### BACKGROUND OF THE INVENTION

Transketolase, a key player in non-oxidative pentose phosphate pathways, shunts carbon away from glycolytic intermediates and forms ribose-5-phosphate required for nucleic acid biosynthesis. Because of its role in ribose synthesis—and thus RNA and DNA synthesis—transketolase may also play a critical role in regulating cell proliferation. Indeed, non-oxidative pentose phosphate pathways are often stimulated in situations of active cell proliferation, such as in tumor cells.

Transketolase (TK) utilizes thiamine (vitamin B₁), converted to thiamine pyrophosphate (TPP) by thiamine pyrophosphokinase, as its co-factor for catalysis. TPP is the active form of the vitamin. Once bound, the TPP co-factor is inaccessible to the solvent and has no appreciable off-rate. Other TPP utilizing enzymes include alpha-ketoglutarate dehydrogenase (kGDH) and pyruvate dehydrogenase (PDH). Glucose-6-phosphate dehydrogenase (G6PDH) can also produce ribose via an oxidative pathway with concurrent generation of two molecules of NADPH. Proper monitoring of these key enzymes would provide a useful indication of the state of cell proliferation and cell growth in tumors.

Compounds that mimic the transketolase co-factor TPP can be potent and long-lasting inhibitors for the transketolase enzyme. The slow off-rate of TPP mimetics from transketolase makes them behave much like irreversible inhibitors. This property provides a convenient enzymatic assay for measuring inhibition of transketolase and TPP utilizing enzymes in blood, tumors and tissues after a dosing regimen.

It would be highly desirable to have improved diagnostic assays with quicker and greater accuracy and/or sensitivity for measuring the activity of transketolase as well as of the other key enzymes of the pentose phosphate pathways (i.e. G6PDH and other TPP utilizing enzymes; PDH and kGDH). Such an assay could also be used as a tool for assessing the potency and selectivity of specific anti-cancer treatments acting on pentose phosphate pathways.

### SUMMARY OF THE INVENTION

The invention provides improved methods for measuring TK, kGDH, PDH and G6PDH activities, e.g., in cultured cells, blood, tumors and other tissues using fluorescence. The increased sensitivity of this fluorescence assay, which can be performed on cell lysates, significantly improves the assays of the present invention over enzymatic assays that monitor absorption. Accordingly, assays of the invention are useful for drug discovery, as well as for preclinical and clinical development of anti-cancer therapeutics and for determining drug dosing and scheduling in the clinic.

### BRIEF DESCRIPTION OF THE DRAWINGS

FIG. 1: An enzymatic reaction for transketolase in the non-oxidative pentose phosphate pathway. Transketolase activity is monitored by GAPDH-catalyzed conversion of NAD to NADH, resulting in the production of fluorescent NADH,

FIG. 2: The enzymatic activities of transketolase (TK) in blood (grey bars) and spleen (black bars) and alpha-ketoglutarate dehydrogenase (kGDH) in brain (white bars) after a single dose of N3-pyridyl thiamine (N3PT) in mice (100 mg/kg, i.p.) from 0 hr to 120 hr after dosing.

FIG. 3: TK activity in various organs (tumor, blood, brain, heart, kidney, lung, spleen) after 11 days of a bi-daily 100 mg/kg dosing treatment with N3PT (white bars), oxythiamine (black bars), or vehicle (grey bars).

FIG. 4: kGDH activity in various organs (tumor, blood, brain, heart, kidney, liver, lung, spleen) after 11 days of a bi-daily dosing treatment with N3PT (white bars), oxythiamine (black bars), or vehicle (grey bars).

FIG. 5: G6PDH activity in various organs (tumor, blood, brain, heart, kidney, liver, lung, spleen) after 11 days of a bi-daily dosing treatment with N3PT (white bars), oxythiamine (black bars), or vehicle (grey bars).

FIG. 6: TK activity in various tested cell lines with (HCT116-TK, HT1080-TK) and without (HCT116, HT1080, DLD-1, HuCa25 (22Rv1)) over expressed TK. A bold horizontal line designates the detection limit. TK activity is plotted as the detected fluorescence (FU/min) as a function of the number of cells.

FIG. 7: kGDH activity with 200 μM TPP in various tested cell lines with and without (HCT116, HCT116-TK, HT1080, HT1080-TK, DLD-1, HuCa25 (22Rv1)) over expressed TK. kGDH activity is plotted as the detected fluorescence (FU/min) as a function of the number of cells.

FIG. 8: G6PDH activity in various tested cell lines with (HCT116-TK, HCT 1080-TK) and without (HCT116, HT1080, DLD-1, HuCa25 (22Rv1)) over expressed TK. G6PDH activity is plotted as the detected fluorescence (FU/min) as a function of the number of cells.

FIG. 9: TK activity in various cell lines expressed as percentage enzymatic activity (the slope of initial linear range) of control wells that were not treated with compounds. The values (y) were plotted as a function of the log concentration (micromolar) (x) and fitted to a sigmoidal dose-response curve.

FIG. 10: Inhibition of TK and kGDH by N3PT in treated cells, expressed as percentage inhibition as a function of the log of compound concentration (micromolar).

FIG. 11: Competitive inhibition of TK by N3PT in treated cells with increasing doses of thiamine, expressed as percentage enzymatic activity (the slope of initial linear range) of control cells that were not treated with compounds. The values (y) were plotted as a function of the log concentration (x) and fitted to a sigmoidal dose-response curve.

FIG. 12: Competitive inhibition of TK by N3PT, expressed as relative mean transketolase activity as a function of the log of N3PT dose (micromolar) at days 2, 3, 5 and 7. Cells were treated with N3PT for two days after plating (day 2) then washed out and activity was monitored thereafter.

### DETAILED DESCRIPTION OF THE INVENTION

Unless otherwise defined herein, scientific and technical terms used in connection with the present invention shall have the meanings that are commonly understood by those of ordinary skill in the art to which this invention pertains. Further, unless otherwise required by context, singular terms shall include pluralities and plural terms shall include the singular.

The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al, Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Worthington Enzyme Manual, Worthington Biochemical Corp. Freehold, N.J.; Handbook of Biochemistry: Section A Proteins, Vol I 1976 CRC Press; Handbook of Biochemistry: Section A Proteins, Vol II 1976 CRC Press; Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000).

The nomenclatures used in connection with, and the laboratory procedures and techniques of, molecular and cellular biology, microbiology, genetics, protein and nucleic acid biochemistry and hybridization, enzymology and medicinal and pharmaceutical chemistry described herein are those well known and commonly used in the art.

All publications, patents and other references mentioned herein are incorporated by reference in their entirety.

The following terms, unless otherwise indicated, shall be understood to have the following meanings:

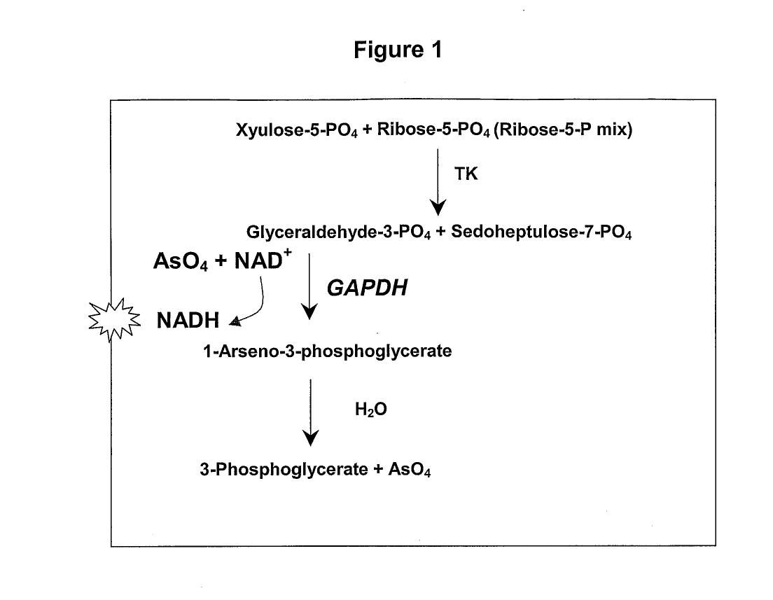

As used herein, the term “transketolase” or “TK” refers to a key enzyme in the non-oxidative pentose phosphate pathway whose activity can be measured by the reaction depicted in FIG. 1.

As used herein, the term “N3PT” or “N3-pyridyl-thiamine” refers to 3-[(2-amino-6-methyl-3-pyridinyl)methyl]-5-(2-hydroxyethyl)-4-methyl thiazolium.

As used herein, the term “activity” refers to a process or action of excitation or inhibition, and encompasses any specific activity of the factor in question (e.g., specific binding to one or more other cellular factors, and enzymatic activity when referring to enzymes, such as TK and other pentose phosphate and TPP utilizing enzymes).

As used herein, the term “pentose phosphate pathways” refers to both oxidative and non-oxidative forms of the pathway.

As used herein, the term “tumor” refers to either a heterogeneous tumor sample from a patient that contains a mass of tumorigenic cells and non-transformed normal cells, or to tumorigenic cells or tumor-derived cells (extracted from a tumor in an animal and cultured separately from the tumor either in vitro or in vivo), or cell lines developed from any of the above. The term encompasses both hard and soft tumors that contain primary or metastatic tumorigenic cells associated with a malignancy. Examples include but are not limited to hematopoietic malignant cells (e.g., lymphomas, leukemias) and other malignant masses derived from specific organs (e.g., fibrosarcomas, adenocarcinomas, hepatomas, and melanomas).

As used herein, the term “drug treatment” refers to the general act of administering or applying a drug to a patient for a disease or injury. This act can include but is not limited to the general manipulation and management of factors such as the dosing, concentration and scheduling of the drug regimen so applied.

As used herein, the term “TPP mimetic” or “TPP mimetic drug” refers to a compound that is similar in both structure and function to a known compound or class of compounds which inhibit thiamine pyrophosphate utilizing enzymes.

As used herein, the term “tumor-derived cell” refers to a cell extracted from a tumor in an animal which has been cultured separately from the tumor, in vitro or in vivo.

As used herein, the term “patient” refers to an animal or a human.

Exemplary methods and materials are described below, although methods and materials similar or equivalent to those described herein can also be used in the practice of the present invention and will be apparent to those of skill in the art. In case of conflict, the present specification, including definitions, will control. The materials, methods, and examples are illustrative only and not intended to be limiting.

Throughout this specification and claims, the word “comprise” or variations such as “comprises” or “comprising”, will be understood to imply the inclusion of a stated integer or group of integers but not the exclusion of any other integer or group of integers.

Assays of the Invention

The assays and methods of the present invention measure the enzymatic activities of TPP utilizing enzymes including enzymes involved in the pentose phosphate pathways. TK activity for example, is known to be elevated in tumorigenic tissues. The assays can be used to monitor the treatments with drugs that act on the pentose phosphate pathway in tumorigenic as well as non-tumorigenic tissues and organs. The assays of the invention are amenable to frequent sampling and measuring of the enzymatic activities in a treated patient over time.

In one embodiment, the invention provides a method for determining the activity of a TPP utilizing enzyme comprising the step of monitoring the production of NADH, e.g., conversion of NAD to NADH, by fluorescence. In various embodiments, the enzyme is transketolase, alpha-ketoglutarate dehydrogenase or pyruvate dehydrogenase. In some embodiments, the activity is measured from homogenized tissue samples without isolating cell components. The samples may be tumors, blood and other tissues, fresh or frozen, without further purification.

The invention also provides a method for determining the activity of an enzyme in an oxidative ribose-5-phosphate generating pathway, comprising the step of monitoring production of NADPH, e.g., conversion of NADP to NADPH, by fluorescence. In some embodiments, the enzyme is glucose-6-phosphate dehydrogenase (G6PDH).

In either the above-described methods, fluorescence is measured with excitation at about 340±30 nm and emission measured at about 460±30 nm in kinetic mode. In some embodiments, the activity is measured in homogenized cell samples without isolating cell components. In certain embodiments, the samples are tumor cells selected from the group of transformed cell lines, fresh or frozen tumor cells or tissues. The assay is most preferably performed on human tumor cells of any type. Preferably, the assay is performed in multi-well dishes. Preferably, the amount of total protein per assay is less than about 80 micrograms.

The methods of the invention are advantageous over previously used methods because lysate clearance, for example by centrifugation or filtration, is not required. As a result, the assays of the invention are faster and more convenient and avoid possible enzyme loss and degradation during lengthy sample manipulations. Further, because the methods of the invention use fluorescence read out instead of absorption, they are more sensitive. Further, according to the methods of the invention, turbidity and air-bubbles do not affect the reading and thus allow for accurate data acquisition in a more robust and high throughput fashion. The amount of sample required for accurate measurement is very small: (approximately 40 μl of blood and <1 mg of tissue). The small volume requirement of the assays of the invention enables the skilled worker to study precious clinical samples, such as biopsies. Moreover, assays of the invention can be easily adapted to 96-well (or higher) format as described below in the Examples. These features make the assays of the present invention practical for drug discovery, and preclinical and clinical uses.

Assays of the invention may be used to optimize drug dosing in animal studies and during human clinical trials or therapy. Pharmacodynamic (PD) profiling via enzymatic activity is a better indicator for dosing regimen than the traditional pharmacokinetic (PK) profiling. Blood can be used as the most convenient sample for measuring drug exposure and PD properties. When tumor biopsy samples are available, either before, during or after a treatment regimen, they can be used to assess the effectiveness of the treatment. The course of treatment of hematological tumors can be followed by monitoring transketolase activity of the malignant cells. For preclinical studies using animal models, all tissues can be collected to study drug exposure and PD profile, selectivity against other TPP-utilizing enzymes, brain penetrance, and the like.

Monitoring Therapeutic Treatment

The assays and methods of the invention are useful for optimizing therapeutic dosing and/or scheduling of TPP mimetic drugs by sampling and monitoring enzymatic levels of TPP utilizing enzymes in oxidative and non-oxidateive pathways in the treated patient over time. For example, if a TPP mimetic drug, such as N3PT, is administered to a patient to treat a tumor, whole blood and/or a tumor biopsy can be taken from the patient at one or more times after treatment and the enzymatic activities of various TPP utilizing enzymes in the samples measured using one or more assays of the invention (see, e.g., Example 1).

The assays of the present invention, thus, enable the skilled worker to assess the efficacy of the treatment. The assays can also be used to monitor and, if necessary, modify the treatment regimen. This enables the health care provider to monitor and make dosing/scheduling corrections earlier than and in a more systematic manner than with conventional methods for monitoring tumor treatment.

Accordingly, in another embodiment, the invention provides a method for monitoring the effectiveness of a TPP mimetic drug treatment in a in a patient in need thereof, by measuring the activity of a TPP utilizing enzyme in the blood or other tissue of the patient. In preferred embodiments, the method measures the production of NADH by fluorescence: In particularly preferred embodiments, fluorescence is measured with excitation at about 340±30 nm and emission is measured at about 460±30 nm in kinetic mode. In some embodiments, the TPP utilizing enzyme activity is measured in a tumor biopsy or blood sample of the patient before, during or after the drug treatment. In some embodiments, the TPP utilizing enzyme is transketolase, alpha-ketoglutarate dehydrogenase or pyruvate dehydrogenase.

Assays for Identifying TPP Mimetics Having Desirable Therapeutic Properties

The assays of the invention also are useful for identifying inhibitors of TPP utilizing enzymes. The ability of a candidate compound to inhibit the activity of one or more TPP utilizing enzymes can be determined according to the methods of the invention by detecting reduced enzymatic activity in cells or tissues after treatment with the candidate compound. The selectivity of a compound shown to be an inhibitor for a particular TPP utilizing enzyme can be determined by comparing the relative inhibition of other TPP utilizing enzymes. Inhibition of TK by new TPP mimetics can be further characterized using a competitive binding assay in which TK inhibition is reduced by thiamine in a concentration dependent manner, where high levels of thiamine overcome the effect of inhibition by the tested compound. The desirability of the specificity and affinity of TK inhibition by various TPP mimetics can be determined by comparing and contrasting these results with the effects of N3PT in the thiamine competition assay. The results of these assays enable the skilled worker to assess the therapeutic properties of additional known or novel TPP-derived inhibitors in an efficient and reliable manner. The assays and methods of the invention are, therefore, a practical tool for determining the clinical application of known or novel compounds in the treatment of conditions that may benefit from transketolase inhibition, including but not limited to cancer.

Thus, the invention also provides methods for identifying a TPP mimetic drug for use as a therapeutic agent comprising the step of comparing the inhibition of a TPP utilizing enzyme by a test compound with the inhibition by N3PT. In one embodiment, the TPP utilizing enzyme is transketolase. In a preferred embodiment, the inhibition of the TPP utilizing enzyme is determined by monitoring the production of NADH or NADPH by fluorescence. In some embodiments, inhibition of the TPP utilizing enzyme is measured by competitive binding of the candidate compound in the presence of N3PT or thiamine The following are examples which illustrate various aspects of the invention. These examples should not be construed as limiting. The examples are included for the purposes of illustration only.

### EXAMPLE 1

### Method for Determining Enzyme Activity in Blood, Tumors and Other Tissues

Blood samples from mice were taken (40 μl whole blood) serially or from mice sacrificed at the end of the experiment. Tissues, such as tumors (e.g. from implantation, xenograft, or arising spontaneously), brain, heart, kidney, liver, lung and spleen were taken and flash frozen in liquid nitrogen or on dry ice and stored in −80° C. until time of assay. Four volumes of cold lysis buffer (20 mM HEPES, pH 7.5, 1 mM EDTA, 0.2 g/l Triton X-100® and 0.2 g/L sodium deoxycholate, supplemented with 1 mM DTT and 1 mM PMSF just before use) were added to the blood, which was dissolved by vortexing. For blood that was frozen before coagulation, a clear lysate was obtained. If coagulation occurred, homogenization was used as described below for tissue. Tissues were suspended in lysis buffer in 10 ml round bottom tubes and homogenized with PowerGen 125® (Fisher Scientific) for four seconds at highest power while submerged in an ice-water bath. This step was repeated after a few seconds, as necessary. The lysate was used right away or else flash frozen and stored at −80° C. until assay time. Eighty microliters (80 μl) of lysate (containing 5 mg tissue/ml lysate for TK and kGDH, 0.5 mg/ml for G6PDH) were put directly in individual wells of a 96-well clear-bottomed assay plate. Twenty microliters (20 μl) of 5× reaction mix was added to initiate each enzymatic reaction.

Each reaction was monitored for the appearance of NADH (for transketolase, alpha-ketoglutarate dehydrogenase, pyruvate dehydrogenase) or NADPH (for glucose-6-phosphate dehydrogenase) at room temperature in a fluorescent plate reader with excitation at 340 nM and emission at 470 nM in kinetic mode. The velocity was determined by the slope of the initial linear range of increasing fluorescence, expressed in fluorescent units (FU)/min. The protein concentration in the lysate was determined using Bradford assays (BioRad) with BSA as a standard. The enzymatic velocity was normalized by the total protein concentration for inter-sample comparison. To obtain reliable results across samples, similar starting protein concentrations were prepared (within approximately 30%) so that the normalized velocity required only small adjustments. This was achieved by weighing the tissue and putting in lysis buffer so that the suspensions contained the same amount of tissue/ml lysis buffer for all samples.

The 5× reaction mixes for each enzyme were as follows:

For TK, 15 μl of 5× assay buffer containing final concentrations of 50 mM HEPES, 40 mM KCl, 2.5 mM MgCl2, 5 mM NaArsenate, 1 mM NAD, 2 unit/ml glyceraldehydes-3-phosphate dehydrogenase (GAPDH) was added to 80 μl of lysate. Reaction kinetics were monitored on a fluorescent plate reader to allow any possible background activity via GAPDH to burn out. Then 5 μl of substrate mix containing final concentrations of 0.5 mM ribose-5-phosphate and 0.5 mM xylulose-5-phosphate was added to initiate the reaction. The reaction kinetics were monitored using a fluorescent plate reader, and the slope of the initial linear range was recorded as the velocity of the reaction (FU/min). See FIG. 1 for the reaction scheme.

For alpha-ketoglutarate dehydrogenase (kGDH), the final concentrations were: 50 mM HEPES, pH 7.5, 40 mM KCl, 2.5 mM MgCl₂, 5 mM DTT, 2 mM a-ketoglutarate acid, 0.5 mM NAD, 0.15 mM CoA. The reaction scheme is:

### kGDH

Alpha-ketoglutarate+CoA+NAD→succinyl-CoA+CO₂+NADH

For pyruvate dehydrogenase (PDH), the final concentrations were: 50 mM HEPES, pH 7.5, 40 mM KCl, 2.5 mM MgCl₂, 5 mM DTT, 2 mM pyruvate, 0.5 mM NAD, 0.15 mM CoA. The reaction scheme is:

### PDH

Pyruvate+CoA+NAD→acetyl-CoA+CO2+NADH

For glucose-6-phosphate dehydrogenase (G6PDH), the final concentrations were: 50 mM Tris-HCl, pH 8.1, 1 mM MgCl₂, 1 mM DTT, 0.2 mM NADP, 0.5 mM glucose-6-phosphate. The reaction scheme is:

### G6PDH

G6P+NADP→6-phosphoglucono-lactone+NADPH
One more NADPH is generated from further oxidation of 6-phosphoglucono-lactone by enzymes present in the cells.

### EXAMPLE 2

### Method for Determining In Vivo Enzyme Inhibition

To investigate the amount of in vivo enzyme inhibition following treatment with an inhibitor, we administered a single dose of N3PT (100 mg/kg, intraperitoneally [i.p.]) and blood, spleen and brain samples were collected at various times from 0-120 hours after dosing. The enzymatic activities were determined as described in Example 1. TK activity was measured in blood and spleen. Because TK activity in the brain is below the detection limit of the assay, kGDH activity was measured in brain samples. Measurements were taken at various time intervals

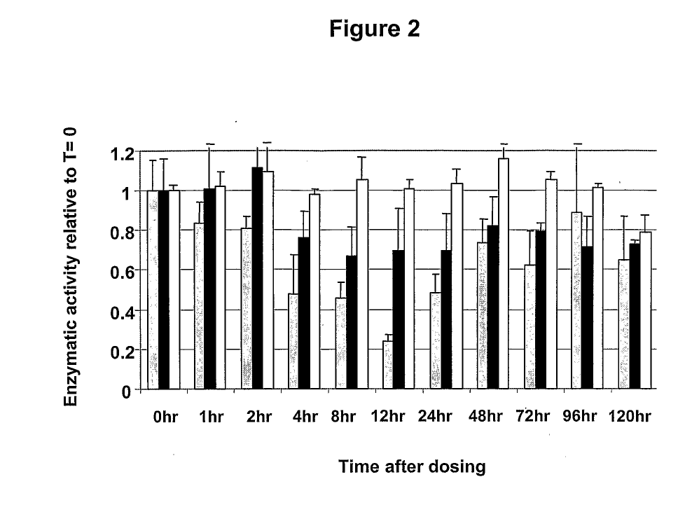

As shown in FIG. 2, using the above-described assay, inhibition of transketolase activity in the blood was readily detectable with 80% inhibition being detected at the nadir (12 hours) (FIG. 2). Transketolase inhibition was detectable in this assay at 1 hour post-treatment and was still evident at the final sampling at 120 hours (5 days). Inhibition of transketolase activity of up to 35% at 8-24 hours post-treatment was detected in the spleen. Brain transketolase activity was below the detection limit. No significant inhibition of brain kGDH activity was observed, indicating that N3PT has little or no significant brain penetration after a single dose.

We measured enzyme activity in an expanded panel of tissues from mice treated with N3PT or OxyT for 11 days i.p., twice per day. Samples were taken from tumors, blood, brain, heart, kidney, lung and spleen, and TK, kGDH and G6PDH activities were determined as described above.

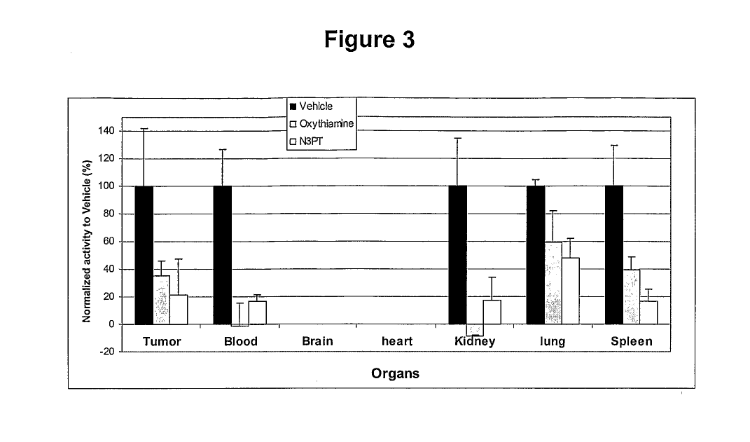

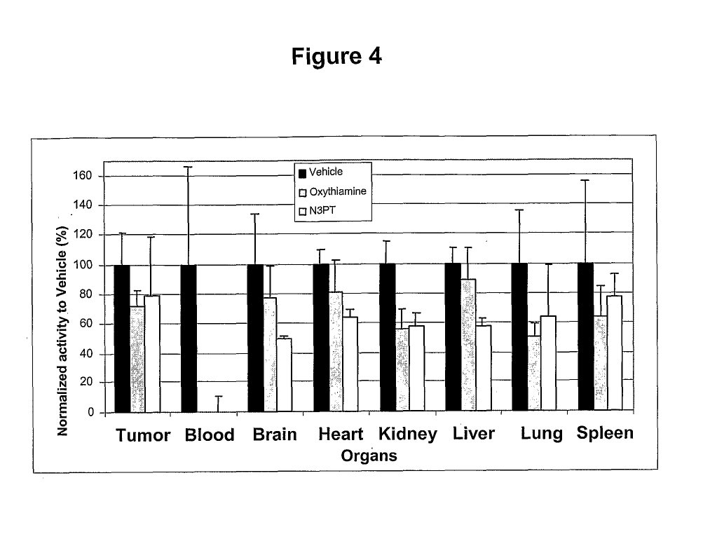

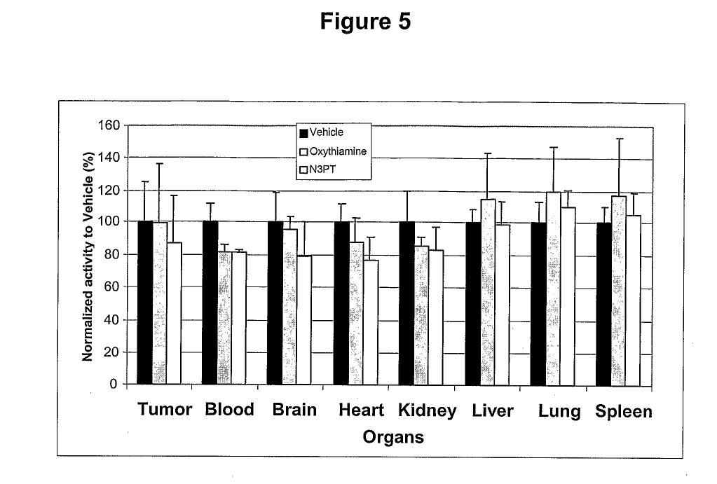

As shown in FIG. 3, we detected the percent inhibition of TK activity in tumors after N3PT dosing (70%), and after OxyT (40%), both as compared to control (vehicle only) using the above-described assay. Inhibition was most pronounced in blood and kidney, followed by spleen and lung. By contrast, liver enzymatic activity was hardly affected. TK levels in the brain and heart were below the detection limit in both experimental conditions as well as in controls. As shown in FIG. 4, kGDH activities were generally less affected than TK except in blood, where it was also obliterated. One can also measure the brain exposure of the compounds via kGDH. These results show that N3PT can inhibit the activity of both TK and kGDH. As G6PDH does not use TPP as co-factor, it was unlikely to be affected directly by TPP mimetics. Indeed, as shown in FIG. 5, no significant decrease in enzymatic activity was observed following treatment with either oxythiamine or N3PT. The G6PDH activity measured, therefore, served as internal controls for sample integrity.

### EXAMPLE 3

### Cell-Based Enzyme Activity Assay

To determine the activity of various TPP utilizing enzymes in human tumor cells and cell lines, we cultured cells including HCT116 (human colon carcinoma cells having an activated K-ras gene, K-ras^{G13D}); HT1080(human fibrosarcoma cells); DLD1 (colon adenocarcinoma), Rv22 (prostate carcinoma), HCT115 (colon carcinoma), MIA PA CA-2 (pancreas carcinoma), SK-Mel-5 (melanoma) National Cancer Institute [NCI]) and murine cancer lines: LLC (Lewis lung carcinoma), 4T1(breast carcinoma), and CT26 (colon carcinoma), all available from the American Type Culture Collection [ATCC] unless otherwise specified) in standard conditions (37C, 5% CO₂) in DMEM media (10% FBS, 1% Penicillin-Streptomycin) and harvested at 60-80% confluency by trypsinization. Approximately 2 million cells were collected in Eppendorf tubes and washed 2 times (2×) with PBS by centrifuigation and stored at −20° C. as cell pellets. Ice-cold lysis buffer (1 ml) (20 mM HEPES, pH 7.5, 1 mM EDTA, 0.2 g/l Triton X-100 and 0.2 g/L sodium deoxycholate, supplemented with 1 mM DTT and 1 mM PMSF just before use) was added to the cell pellet. Cells were lysed by vortexing. Lysate containing different amounts of cells were brought up to a volume of 80 μl with lysis buffer and the reactions were carried out as described in Example 1 in a clear bottomed 96-well assay plate.

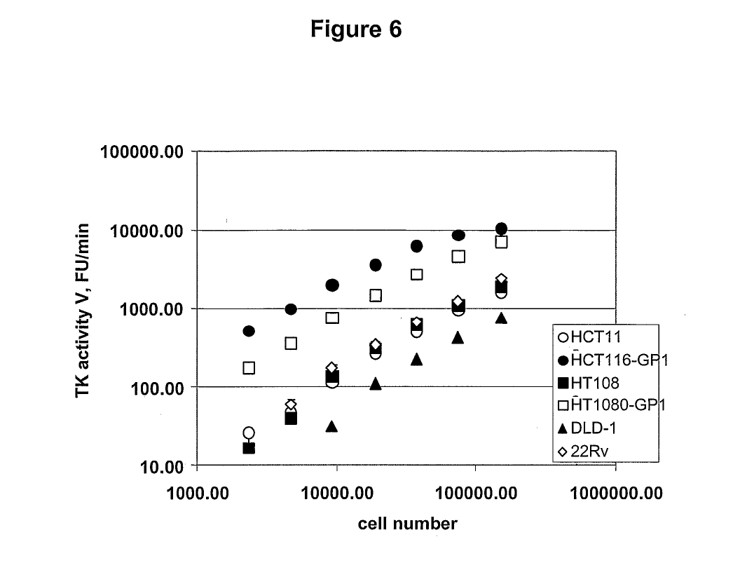

The reactions were monitored in a fluorescent plate reader and enzymatic velocity was determined as described above. TK activities could be measured using lysate from ≧5000 cells in most cell lines tested. However, in cell lines over expressing TK, the detection limit could be as low as 250 cells. FIG. 6 shows the TK activity and the detection limit (in cell number).

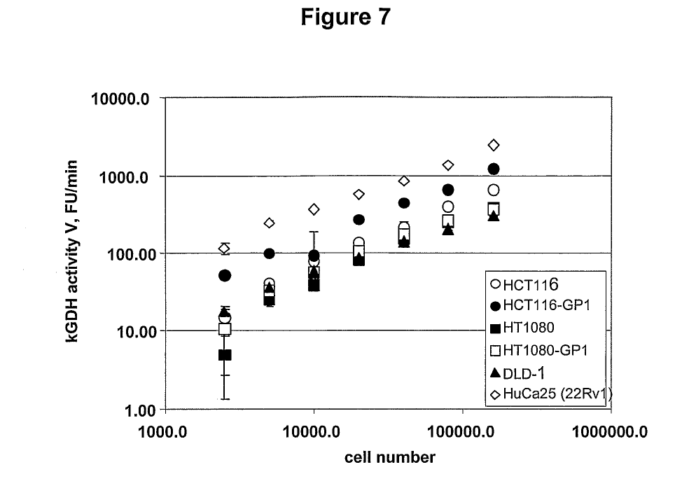

kGDH activity could be determined using ≧10000 cells in most cell lines tested. FIG. 7 shows the KGDH activity in various cell lines with (HCT116-TK, HT1080-TK) and without (HCT116, HT1080, DLD-1, HuCa25 (22Rv1)) over expressed TK. Enzymatic kGDH activity is plotted as the detected fluorescence against the number of cells.

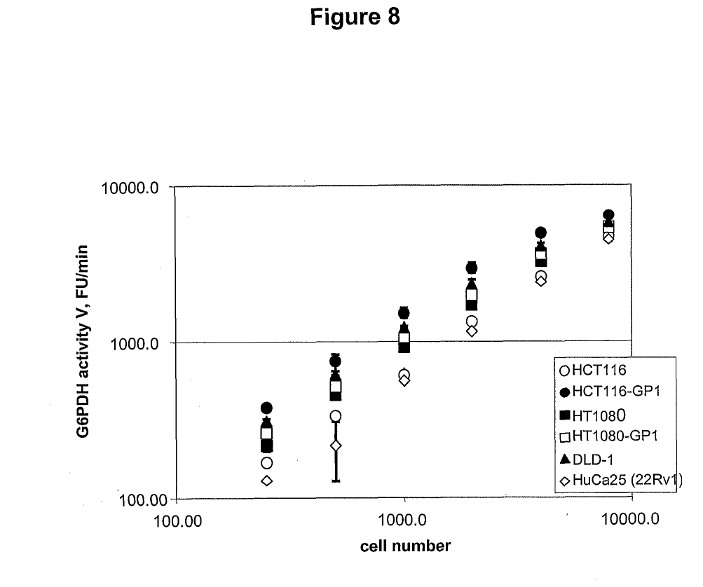

FIG. 8 shows the G6PDH activity. G6PDH had the highest enzymatic activity in the cells, can be determined using as little as 250 cells.

The enzymatic activities could also be measured in 96-well plates directly. Adherent cells were plated in clear-bottomed 96-well cell culture plates, allowed to attach and grown until the cell density reached about 60-80% confluency. Plates were inverted to remove media and blotted on paper towels to remove residual media. Plates were stored at −20° C. or immediately assayed for enzymatic activity. 80 μl of lysis buffer was added to each well, and the cells incubated at RT for 10 min to allow cell lysis. Reactions were carried out and monitored as described above.

### EXAMPLE 4

### Determining Inhibition Constants of TPP Mimetics for TK, kGDH and PDH Activities

We determined the IC₅₀ of compounds that inhibit the activity of the TPP utilizing enzymes TK, kGDH and PDH in a convenient 96-well format using an optimized version of the method described in the previous Examples.

The high thiamine levels in conventional cell culture media (e.g., DMEM (12 μM), RPMI (3 μM)) masks the inhibitory effect of such inhibitors. To measure the IC₅₀ of such inhibitors, thus, a thiamine depleted DMEM, containing all the ingredients of normal DMEM except for thiamine (HyClone, custom order), was used. Thiamine-depleted media (TDM) is made up with thiamine-depleted DMEM, 10% FBS, which contains negligible amount of thiamine (estimated to be ˜3-5% nM) and 1% Penicillin-streptomycin.

Log-phase growing cells were trypsinized and resuspended in TDM. Optimization of the initial cell counts is recommended for each cell line to ensure that enzyme activity can be observed at the beginning and that healthy growth can continue. For cell lines that have a doubling time of approximately 24 hours and cellular transketolase levels high enough to be reliably detected with five thousand (5K) cells such as HCT116 and HT1080, 8K cells are recommended. Using this guide, IC₅₀s could be monitored for 6 days and 2-4 days of treatment resulted in a value that was stable and reproducible.

Media containing 8K cells (95 μl) were used to seed individual wells in a 96-well clear-bottom cell cultured treated sterile plate. Inhibitor compounds were dissolved in 100% DMSO as 10 mM solutions. Serial dilutions were then made in 100% DMSO to make up a 100× stock, then diluted to 20× in double-deionized H2O (ddH2O). Twenty-four hours after seeding, 5 μl of 20× inhibitor compound stock solution was added to the cells so that the final concentration of DMSO is 1%. Media were changed after 24 hours. Forty-eight hours after inhibitor compound treatment, the plates were inverted to remove media and blotted on a paper towel. The plates were then either subjected to enzymatic reactions immediately or were frozen at −20° C. for future assays. Enzymatic reactions were carried out as described in the previous Examples. Enzymatic inhibition was expressed as percent of control wells that were not treated with compounds. The values (y) were plotted as function of the log concentration (x) and fitted to a sigmoidal dose-response curve with variable slopes that bears the equation: y=bottom+(top−bottom)/(1+10ˆ((logEC50−x)*hillslope)).

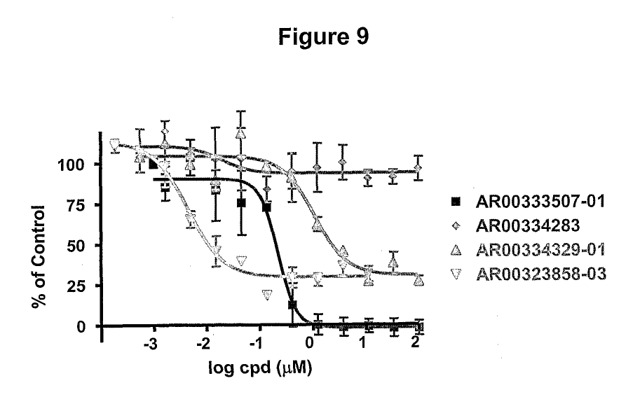

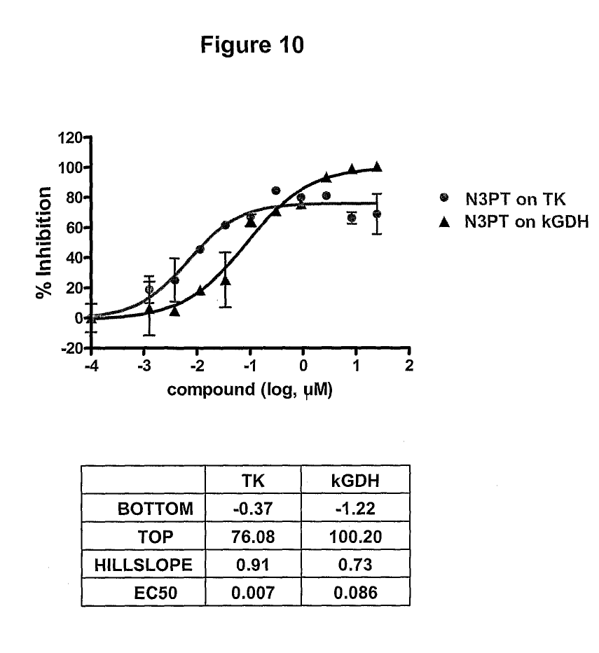

Results shown in FIG. 9 indicate that the slow off-rate of the inhibitor compounds preserves enzymatic inhibition in cell lysates. Results shown in FIG. 10 indicate that the IC₅₀ of N3PT for both TK and kGDH was 20 nM and 86 nM respectively. Therefore, N3PT inhibits the activity of both TK and kGDH but is a more potent inhibitor for TK than kGDH.

### EXAMPLE 5

### Cell-Based Enzyme Assay for Inhibitors of Substrate Binding

Inhibitors for the substrate binding site are generally reversible with a fast off-rate. Dilution with assay buffers and the use of the detergents affect the enzyme-inhibitor complex and are, thus, unsuitable for an assay involving substrate inhibitors. To determine the effects of these types of inhibitors, a new method was devised. Xylulose has been shown to cause a temporary increase in sedoheptulose-7-phosphate in hepatocytes (Vincent et al. “D-xylulose-induced depletion of ATP and Pi in isolated rat hepatocytes”. FASEB J., 3:1855-1861 (1989)).

Human tumor cells and cell lines were first cultured as described in Example 3 and plated in normal media. The following day, xylulose was added to each use to achieve a final concentration of 1 mM. The media was then removed and 50 μl of 50% acetylnitrile solution was added to the cells and the amount of sedoheptulose-7-phosphate was quantified by liquid chromatography mass spectrometry (LCMS). IC50 values were derived in the same fashion as described in previous Examples, except the relative amount of sedoheptose-7-phosphate is used rather than velocity of enzymatic activity.

### EXAMPLE 6

### Method for Characterizing N3PT as a Competitive Inhibitor of Transketolase

HCT116 cells were plated in clear-bottomed 96-well culture plates, at a density of 8000 cells/well in TDM. The following morning, N3PT and different amounts of thiamine (12 μM, 2.4 μM, 0.48 μM, 0.096 μM, 0.0192 μM, 0 μM) were added to the cells. Forty-eight hours later, plates were inverted to remove media and blotted on paper towels to remove residual media. Plates were immediately assayed for enzymatic TK activity. 80 μl of lysis buffer was added to each well, and the cells incubated at RT for 10 min to allow cell lysis. After lysis, 20 μl of the aforementioned 5× reaction mix was added to each well to initiate the reaction. Reactions were monitored as described above.

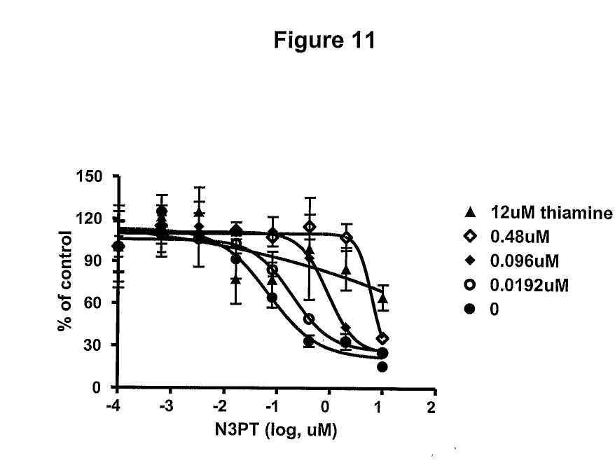

The inhibitory effect of N3PT on TK activity was measured in the presence of increased concentrations of thiamine, as shown in FIG. 11. Inhibition of TK by N3PT was reduced by thiamine in a concentration dependent manner, where high levels of thiamine (e.g., 12 μM) were shown to overcome N3PT inhibition.

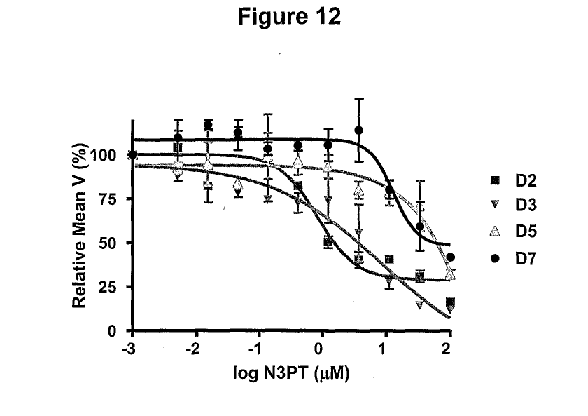

Additional time course experiments revealed that the inhibitory effect of N3PT on cellular TK activity persisted for several days, as depicted in FIG. 12. Cells were plated in TDM media 24 hours earlier, different concentration of N3PT and 100 nM thiamine were added to the cells and treated for 48 hour to allow N3PT to exert its inhibitory effect (day 2; d2). The media was then changed to contain only 100 nM thiamine and one plate was assayed every day for the next five days (d3 to d7). At the end of study on day 7 (d7), TK activity had not fully recovered. These results demonstrate that the inhibitory effect of N3PT can persist for many days.

What is claimed is:

1. An assay for determining the activity of a TPP utilizing enzyme comprising the step of monitoring production of NADH by fluorescence.

2. The assay of embodiment 1, wherein the TPP utilizing enzyme is transketolase, alpha-ketoglutarate dehydrogenase or pyruvate dehydrogenase.

3. The assay of embodiment 1, wherein the activity is monitored by GAPDH-catalyzed conversion of NAD to NADH, resulting in the production of fluorescent NADH,

4. The assay of embodiment 1, wherein the activity is measured from homogenized cell samples without isolating cell components.

5. The assay of embodiment 4, wherein the samples are tumors, blood and tissues, fresh or frozen, without further purification.

6. An assay for determining the activity an enzyme in an oxidative ribose-5-phosphate generating pathway, comprising the step of monitoring production of NADPH by fluorescence.

7. The assay of embodiment 6, wherein the enzyme is glucose-6-phosphate dehydrogenase.

8. The assay of embodiment 6, wherein the activity is monitored by G6PDH-catalyzed conversion of NADP to NADPH, resulting in the production of fluorescent NADPH,

9. The assay of embodiment 6, wherein activity is measured from homogenized cell samples without isolating cell components.

10. The assay of embodiment 9, wherein the samples are tumor cells selected from the group of transformed cell lines, fresh or frozen tumor cells or tissues.

11. The assay of embodiment 1 or 6, performed on human tumor cells.

12. The assay of embodiment 1 or 6, performed in multi-well dishes.

13. The assay of embodiment 1 or 6, wherein the assay is cell-based.

14. The assay of embodiment 1 or 6, wherein fluorescence is measured with excitation at about 340±30 nm and emission measured at about 460±30 nm in kinetic mode.

15. The assay of embodiment 1 or 6, wherein the amount of total protein per assay is less than about 80 micrograms.

16. A method for monitoring the effectiveness of a TPP mimetic drug treatment in a cancer patient, comprising the step of measuring the activity of a TPP utilizing enzyme by monitoring the production of NADH by fluorescence.

17. The method according to embodiment 16, wherein the activity is measured by sampling a tumor biopsy or whole blood of the patient before, during or after said drug treatment.

18. The method according to embodiment 16, wherein the measurement of said activity can be used to modify said drug treatment in the patient.

19. The method according to embodiment 16, wherein the drug treatment is optimized through recurring measurements of said activity in a patient.

20. The method according to embodiment 16, wherein the TPP utilizing enzyme is transketolase, alpha-ketoglutarate dehydrogenase or pyruvate dehydrogenase.

21. The method according to embodiment 16, wherein the activity is monitored by GAPDH-catalyzed conversion of NAD to NADH, resulting in the production of fluorescent NADH,

22. The method according to embodiment 16, wherein fluorescence is measured with excitation at about 340±30 nm and emission measured at about 460±30 nm in kinetic mode.

23. A method for identifying a TPP mimetic drug for use as a therapeutic agent comprising the step of comparing the inhibition by N3PT of a TPP utilizing enzyme with the inhibition by a test TPP mimetic drug.

24. The method according to embodiment 23, wherein the inhibition of the TPP utilizing is determined by monitoring the production of NADH by fluorescence.

25. The method according to embodiment 24, wherein the activity is monitored by GAPDH-catalyzed conversion of NAD to NADH, resulting in the production of fluorescent NADH.

26. The method according to embodiment 23, wherein the inhibition of the TPP utilizing enzyme is measured by competitive binding in the presence of N3PT or thiamine.

27. The method according to any one of embodiments 23-26, wherein the TPP utilizing enzyme activity is transketolase.
